(19) United States
(12) Patent Application Publication
(10) Pub. No.: US 2010/0185213 A1
(43) Pub. Date: Jul. 22, 2010

(54) Hair removal apparatus

(76) Inventor: Phillip L. Lam, Monterey Park, CA (US)

Correspondence Address: LEONARD TACHNER, A PROFESSIONAL LAW;CORPORATION, 17961 SKY PARK CIRCLE, SUITE 38-E, IRVINE, CA, 92614, US

(21) Appl. No.: 11/887,178

(22) PCT Filed: Mar. 24, 2006
(86) PCT No.: PCT/US2006/011089

§ 371 Date: Sep. 25, 2007

Related U.S. Application Data
(60) Provisional application No. 60/665,529, filed on Mar. 25, 2005.

(51) Int. Cl. A61B 17/50

(52) U.S. Cl. 606/131

(57) ABSTRACT

An apparatus (10) for removing hair employs two overlapping thread portions (14) and (16) wherein one thread portion is helically wound or coiled around the other to form at least one hair trap (18) to grasp and pull strands of hair. A motorized oscillation arm (32) alternately tightens and loosens each side of one thread portion to manipulate the hair trap. The thread is held in tension in a readily removable and replaceable yoke (12) to facilitate disposal of the used thread and replacement with unused thread. A housing (20) contains a battery-operated motor (24) to move the oscillation arm which has hooks (36) to engage the thread.

### CROSS-RELATED APPLICATIONS

This invention claims priority from U.S. Provisional Patent Application Ser. No. 60/665,529 filed Mar. 25, 2005.

### FIELD OF THE INVENTION

This invention relates to a hair removal apparatus which employs a pair of threads, one straight and the other coiled around the straight one in a manner to form a linear arrangement of tweezer-like hair traps when the twisted thread is rolled in alternative directions about the straight thread to translate the twisted thread portion by a motorized actuation.

### BACKGROUND ART

Hair removal apparatus including two helicoils interwound to define hair traps are well known in the art. U.S. Pat. No. 6,743,238 issued Jun. 1, 2004 discloses one such apparatus. U.S. Pat. No. 5,951,573 issued Sep. 14, 1999 describes another. The art is replete with numerous such examples and apparently is based on ancient Chinese techniques. The most relevant prior art patent appears to be U.S. Pat. No. 4,983,175 wherein two mutually twisted elongate loops create a tweezer-like action in the twisted portion which remains relatively stationary.

Unfortunately, all such apparatus uncovered by the applicant are awkward to use, relatively expensive and not disposable. Moreover, the U.S. Pat. No. 4,983,175 device although also motorized, is difficult to accurately locate precisely next to hair to be removed, uses a relatively complicated mechanism, is not readily configured for disposing and replacing used threads and raises safety concerns.

### SUMMARY OF THE INVENTION

The invention is based on the recognition that a simple and inexpensive hair removal apparatus may be achieved by winding one thread about another and manipulated a thread in a simple manner to achieve the requisite hair removal action. Moreover, the resulting apparatus would be sufficiently inexpensive to be disposable.

In accordance with another aspect of this invention, the “disposable” hair removal apparatus herein is configured to move with a powered driver to engage the thread in a manner to alternatively tighten and loosen the linear arrangement of hair traps defined by the interwound threads while permitting accurate placement thereof immediately adjacent the hair which it is desired to remove.

A key feature of the preferred embodiment is a removable and replaceable thread bearing yoke, which holds two threads in tension, one of which is relatively straight and one of which is twisted about the straight one to form a hair trapping linear configuration which is caused to translate by alternatively tightening and loosening the wound thread on opposite ends of the hair trapping portion. This alternate tightening and loosening causes the hair trapping portion to linearly translate back and forth to grasp and remove short hairs near their roots and is facilitated by a motorized rotating disc and connecting arm which, in turn, is connected to an oscillating arm which engages the twisted thread on opposite ends of the hair trapping portion.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1 is a three-dimensional schematic illustration of the preferred embodiment;

FIGS. 2 and 3 are front and side views respectively, of the preferred embodiment of a hair removal apparatus in accordance with the principles of this invention; and

FIG. 4 is a plan view of the trapping portion of the threads used in the preferred embodiment of FIGS. 1-3.

### DETAILED DESCRIPTION OF A PREFERRED EMBODIMENT OF THE INVENTION

Referring to the accompanying figures, it will be seen that the preferred embodiment of the invention 10 comprises a bow-shaped yoke 12 which holds a first string or thread 14 and a second string or thread 16. The first string is-held at its ends across the upper end of the yoke and the second string is held at its ends at the lower end of the yoke. As shown in FIG. 4, the second string is twisted or rolled around the first string at about their respective middle overlapping sections to form a hair trapping area 18. This hair trapping area is caused to translate along the first string by tensioning the second string on either side of the hair trapping area. If this tensioning is done first on one side of the hair trapping area and then one the other side in an alternating fashion, the hair trapping area will translate back and forth in a linear oscillating motion which can be readily employed to grasp and pull out hairs.

The yoke, including the first and second strings is releasably attached to a housing 20 having a plurality of batteries 22 and a battery-operated motor 24 controlled by an on/off switch 26. The motor's shaft is affixed to a disc 28 which is caused to rotate in one direction by the shaft.

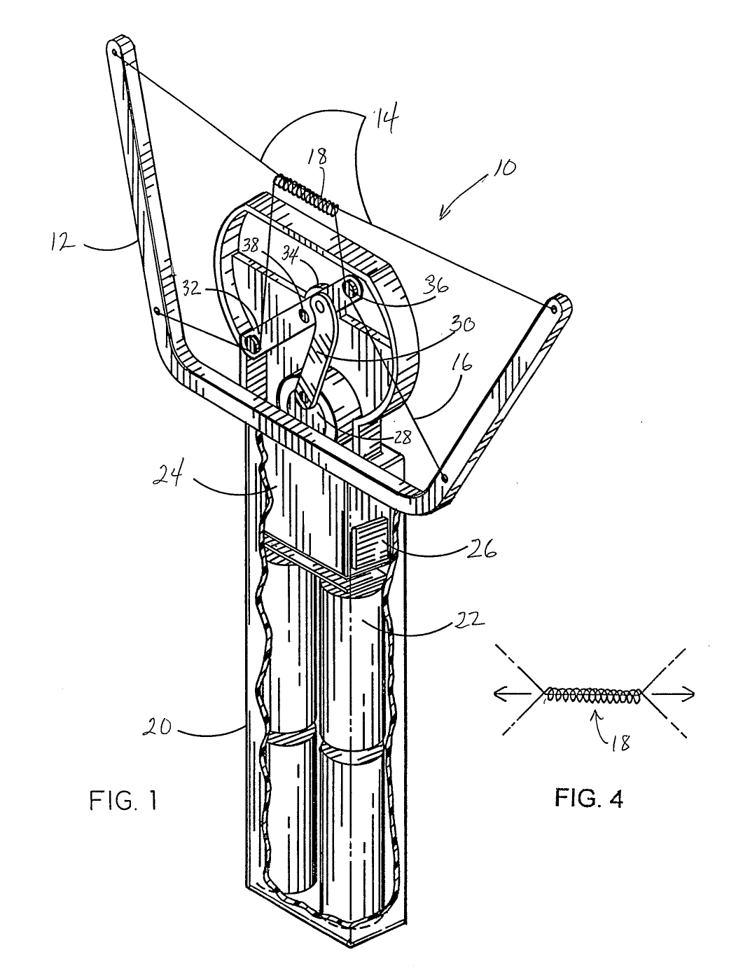

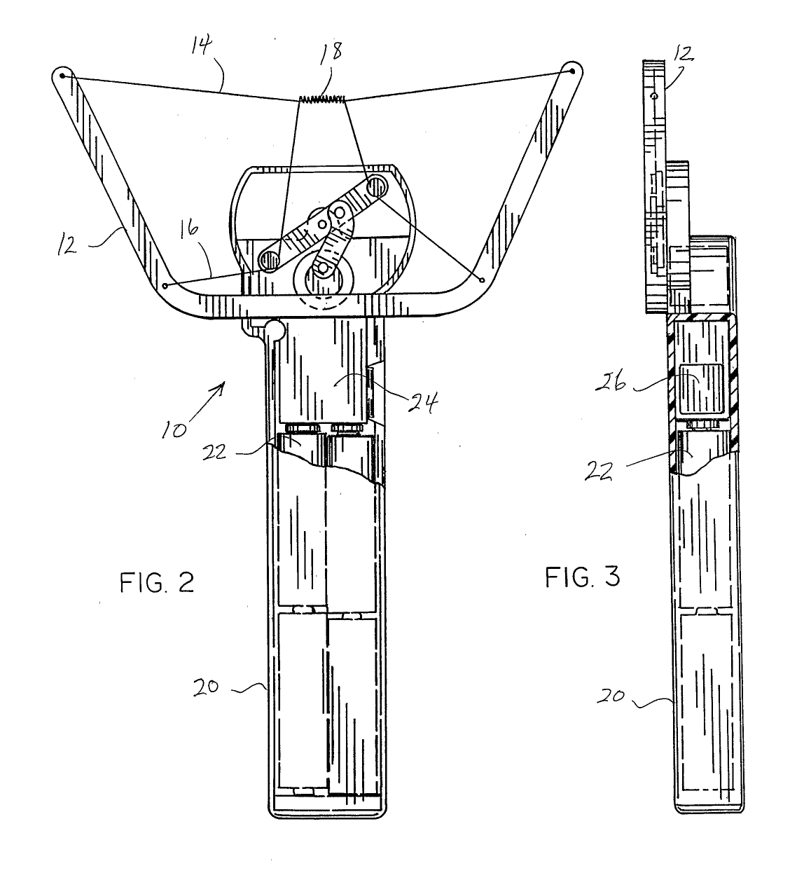

A connecting arm is 30 rotatably affixed at one end to the disc at a location that is radially displaced from the shaft so that that end of the arm will rotate with the disc. Another end of the connecting arm is rotatably connected to an oscillating arm 32 at a location displaced from the oscillating arm's center which, in turn, is rotatably supported by a bracket 34 within the upper portion of the housing. The opposed ends of the oscillating arm have string hooks 36 for engaging the tensioned second string on opposite sides of the hair trapping area as shown best in FIGS. 1 and 2. It will be recognized that the relative positions and interconnections of the disc, connecting arm and oscillating arm will cause the oscillating arm to tilt back and forth about its center or fulcrum 38 with rotation of the disc. The extent of angular travel and speed of the oscillating arm in each direction, (i.e., clockwise and counter-clockwise) will depend upon the rate of rotation and relative size of the disc, length and shape of the connecting arm, and points of connection of the connecting arm with the disc as well as of the connecting arm with the oscillating arm. In the illustrated embodiment, it has been determined that the preferred angular travel of the oscillating arm be about 45° and the oscillating rate be about 2.5 oscillations per second. The goal is to provide sufficient tensioning of the second string on each side of the hair trapping area to linearly translate the overlapped portion of threads at least 0.5 inches to 1.0 inches in each direction away from the center of the first string so that there is adequate motion to grasp and remove hairs in one direction of travel and then release the removed hairs in the opposite direction of travel. It will be understood that the translation of the hair trapping area is actually concurrent with the rotation of the twisted portion of the second string around the first string which thereby creates the tweezer effect to grasp and remove hairs.

It is especially advantageous in the preferred embodiment to employ a bow-shaped yoke which is made of a suitable plastic configured to releasably mate with the housing. This feature makes it both easy and economically feasible to remove and dispose of used threads by replacing the threaded yoke rather than by requiring a “re-stringing” of the yoke. The latter is less desirable because it is time consuming and may alter the amount of tension in the strings which would affect the performance of the apparatus. It is also advantageous to provide a hair trapping area which is positioned beyond the housing profile (as seen best in FIGS. 2 and 3) to permit accurate positioning of the hair trapping area relative to the hairs to be removed without requiring moving mechanical parts to be placed against the skin. This feature makes the illustrated embodiment both more convenient and safer to remove facial hair such as eyebrow hair and the like.

Having thus disclosed a preferred embodiment, it will now be apparent that there may be numerous modifications which could be made without departing from the spirit of the invention or from its inventive features. By way of example, the first and second threads could readily be formed using just one thread configured as a single loop having overlapping portions. Accordingly, the scope of protection is to be limited only by the appended claims and their equivalents.

I claim:

1. Hair removal apparatus comprising first and second threads overlapping in a coiled engagement to define at least one hair trap, said apparatus including a yoke for maintaining said first and second threads resiliently under tension, said apparatus also including a motorized oscillation arm to alternatively tighten and loosen said second string to manipulate said at least one hair trap in a manner to capture and extract hair.

2. Hair removal apparatus as in claim 1 wherein said first and second threads are defined by the single thread which is formed in a loop and coiled to define said hair trap, said yoke including a flexible U-shaped, resilient member, ends of a first side of said loop being anchored at end points of said resilient member, ends of a second side of said loop being anchored at said flexible U-shaped member at positions spaced apart from said end points.

3. Hair removal apparatus as in claim 1, said yoke comprising a flexible U-shaped member having first and second end anchor points, said first thread being anchored between said first and second end anchor points under tension, said second thread being anchored to said U-shaped member at first and second positions on said U-shaped member spaced apart from said first and second end anchor points respectively.

4. Hair removal apparatus as in claim 1 further comprising a housing having a motor and a source of power for said motor, said motor being coupled to said oscillation arm for angular oscillatory motion of said arm.

5. Hair removal apparatus as in claim 4 wherein said motor is coupled to said oscillation arm by a rotating disc and a connecting arm, said connecting arm being connected to said disc and to said oscillation arm.

6. Hair removal apparatus recited in claim 1 wherein said hair trap comprises a portion of said first thread around which a portion of said second thread is helically wound.

7. Hair removal apparatus recited in claim 6 wherein said hair trap manipulation causes the helically wound portion of said second thread to translate along said first thread.

8. Hair removal apparatus recited in claim 1 wherein said first and second threads are two distinct portions of one continuous thread.
